(19) United States
(12) Patent Application Publication
(10) Pub. No.: US 2020/0294632 A1
(43) Pub. Date: Sep. 17, 2020

(54) Medical Data De-Identification

(71) Applicant: Vipin Namboodiri, Bangalore (IN)

(72) Inventor: Vipin Namboodiri, Bangalore (IN)

(73) Assignee: Ricoh Company, Ltd., Tokyo (JP)

(21) Appl. No.: 16/355,161

(22) Filed: Mar. 15, 2019

(51) Int. Cl. G16H 10/60; G16H 10/40; G16H 15/00; G06F 21/62

(52) U.S. Cl. CPC G16H 10/60; G16H 10/40; G16H 20/10; G06F 21/6254; G16H 15/00

(57) ABSTRACT

A system and method for de-identification of electronic medical data is disclosed. The techniques include receiving electronic medical information for storing in an electronic medical record associated with a patient, dividing the electronic medical information into personally identifiable information and health record information, transmitting the health record information to a health record server, scrambling the personally identifiable information, dividing the personally identifiable information into a plurality of chunks of personally identifiable information, creating a plurality of data packages based on the plurality of chunks of personally identifiable information, and transmitting the plurality of data packages to a plurality of identity provider servers, wherein each data package is stored on a separate identity provider server.

### BACKGROUND

### Field of the Invention

The specification generally relates to secure storage of electronic medical record information. In particular, the specification relates to a system and method for separately storing personally identifiable information and health record information associated with a patient.

### Description of the Background Art

Electronic medical records are increasingly being used to store patient data. Electronic medical records provide a large corpus of medical information that can be analyzed for research, etc. However, health privacy laws require specific steps be taken to protect the identity of patients and their medical information.

### SUMMARY

The techniques introduced herein overcome the deficiencies and limitations of the prior art, at least in part, with a system and method for securely storing electronic medical record information. In one embodiment, the system includes one or more processors and a memory storing instructions which when executed cause the one or more processors to receive electronic medical information for storing in an electronic medical record associated with a patient, divide the electronic medical information into personally identifiable information and health record information, transmit the health record information to a health record server, scramble the personally identifiable information, divide the personally identifiable information into a plurality of chunks of personally identifiable information, create a plurality of data packages based on the plurality of chunks of personally identifiable information, and transmit the plurality of data packages to a plurality of identity provider servers, wherein each data package is stored on a separate identity provider server.

Other aspects include corresponding methods, systems, apparatuses, and computer program products for these and other innovative aspects.

The features and advantages described herein are not all-inclusive and many additional features and advantages will be apparent in view of the figures and description. Moreover, it should be noted that the language used in the specification has been principally selected for readability and instructional purposes and not to limit the scope of the techniques described.

### BRIEF DESCRIPTION OF THE DRAWINGS

The techniques introduced herein are illustrated by way of example, and not by way of limitation in the figures of the accompanying drawings in which like reference numerals are used to refer to similar elements.

FIG. 1 is a high-level block diagram illustrating one embodiment of a system for securely storing electronic medical information.

FIG. 2 is a block diagram illustrating one embodiment of a computing device including an electronic medical record interface application.

FIG. 3 is a block diagram illustrating a flow for creating a data package for storing personally identifiable information.

FIG. 4 is a block diagram illustrating one embodiment of a data package for storing personally identifiable information.

FIG. 5 is a flow diagram illustrating one embodiment of an example method for securely storing electronic medical information.

### DETAILED DESCRIPTION

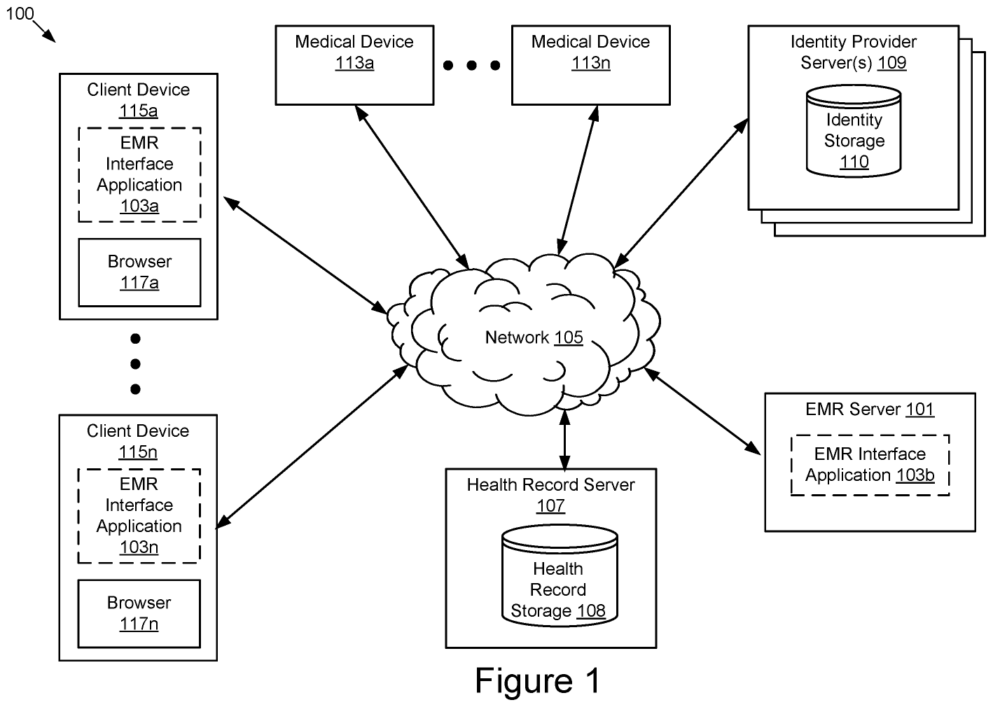

FIG. 1 is a high-level block diagram illustrating one embodiment of a system 100 for securely storing electronic medical information. The illustrated system 100 may have one or more client devices 115a . . . 115n, which can be accessed by users, one or more medical devices 113a . . . 113n, an electronic medical record server 101, a health record server 107, and multiple identity provider servers 109. In FIG. 1 and the remaining figures, a letter after a reference number, e.g., “115a,” represents a reference to the element having that particular reference number. A reference number in the text without a following letter, e.g., “115,” represents a general reference to instances of the element bearing that reference number. In the illustrated embodiment, these entities of the system 100 are communicatively coupled via a network 105.

The network 105 can be a conventional type, wired or wireless, and may have numerous different configurations including a star configuration, token ring configuration, or other configurations. Furthermore, the network 105 may include a local area network (LAN), a wide area network (WAN) (e.g., the Internet), and/or other interconnected data paths across which multiple devices may communicate. In some embodiments, the network 105 may be a peer-to-peer network. The network 105 may also be coupled to or include portions of a telecommunications network for sending data in a variety of different communication protocols. In some embodiments, the network 105 may include Bluetooth communication networks or a cellular communications network for sending and receiving data including via short messaging service (SMS), multimedia messaging service (MMS), hypertext transfer protocol (HTTP), direct data connection, WAP, email, etc. Although FIG. 1 illustrates one network 105 coupling the client devices 115, the medical devices 113, the electronic medical record server 101, the health record server 107, and the identity provider servers 109, in practice one or more networks 105 can be connected to these entities.

In some embodiments, the electronic medical record server 101 may be either a hardware server, a software server, or a combination of software and hardware. The electronic medical record server 101 may be, or may be implemented by, a computing device including a processor, a memory, applications, a database, and network communication capabilities. In general, the electronic medical record server 101 may be health a care server administered by a health care organization for collecting, storing, managing, and transmitting electronic health information of patients.

In the example of FIG. 1, the components of the electronic medical record server 101 are configured to implement an electronic medical record interface application 103b as described in more detail below. In some embodiments, the electronic medical record server 101 may be a cloud-based (e.g., web-based) health care server associated with servicing requests from a client device 115 via the browser 117 or electronic medical record interface application 103a. In some embodiments, the electronic medical record server 101 may also include a dedicated electronic medical record application or medical practice management software application (not shown) for creating, processing, and storing an electronic medical record including medical history, admissions, discharges, transfers, appointments, allergies, lab results, treatment plans, prescriptions, scheduling information, patient billing information, etc. of a patient. An electronic medical record may be defined as an electronic record of health-related information on an individual that can be created, gathered, managed, and consulted by authorized personnel within a health care organization. In some embodiments, electronic medical record server 101 may store an electronic medical record in a file format, such as Java Script Object Notation (JSON) file format.

In some embodiments, the electronic medical record server 101 sends and receives data to and from other entities of the system 100 via the network 105. For example, the electronic medical record server 101 may send and receive data, including electronic medical record data to and from the client device 115. When the client device 115 transmits information about a patient, the electronic medical record server 101 may update the electronic medical record of the patient as described herein while protecting the integrity of the electronic medical record and the identity of the patient. Although only a single electronic medical record server 101 is shown in FIG. 1, it should be understood that there may be any number of electronic medical record servers 101 or a server cluster.

The client device 115 may be a computing device that includes a memory, a processor, for example a laptop computer, a desktop computer, a tablet computer, a mobile telephone, a smartphone, a personal digital assistant (PDA), a mobile email device, a webcam, a user wearable computing device, a television, or any other electronic device capable of accessing a network 105. The client device 115 provides general graphics and multimedia processing for any type of application. The client device 115 includes a display for viewing information provided by the electronic medical record server 101. The client device 115 includes a browser 117. The browser 117 is code and routines stored in a non-transitory computer-readable memory of the client device 115 and is executed by a processor of the client device 115 for accessing the functionality and data provided by electronic medical record server 101 via the network 105.

While FIG. 1 illustrates two client devices 115a and 115n, the disclosure applies to a system architecture having one or more client devices 115. The client device 115 is adapted to send and receive data to and from the electronic medical record server 101. For example, the client device 115 may be accessed by a patient for querying the server 101 about the patient information stored in the patient's electronic medical record. In another example, the client device 115 may be accessed by authorized personnel (e.g., a registered nurse, a lab technician, a receptionist, etc.) to update or access a patient's information on the electronic medical record server 101.

The medical devices 113 may include, but are not limited to, a stethoscope, a blood pressure meter, a pulse oximeter, a thermometer, an ophthalmoscope, a weight and height scale, an otoscope, a camera, a telecardiology device (e.g. an ECG machine), a telepathology device (e.g. a microscope), a teledermatology device (e.g. a high-resolution camera), a teleradiology device (e.g. an ultrasound machine), etc. associated with one or more health care organizations. Authorized personnel who is trained to use the medical device 113 may obtain the patient's medical information. In some embodiments, the medical device 113 may work with the client device 115 to allow authorized personnel to communicate with other entities of the system 100. For example, the client device 115 receives a report associated with a patient including a medical test result from the medical device 113, and sends the report to the electronic medical record server 101 for updating the electronic medical record of the patient.

The electronic medical record interface application 103 may include software and/or logic to provide the functionality for storing and securing patient electronic medical records. For example, the electronic medical record interface application 103 may provide a user interface on client device 115 (e.g., via browser 117 or a native electronic medical record interface application 103) for a user to interact with an electronic medical record of a patient. Additionally, the electronic medical record interface application 103 securely stores personally identifiable information of a patient and health record information of the patient as described in more detail below. In some embodiments, the electronic medical record interface application 103 can be implemented using programmable or specialized hardware, such as a field-programmable gate array (FPGA) or an application-specific integrated circuit (ASIC). In some embodiments, the electronic medical record interface application 103 can be implemented using a combination of hardware and software. In other embodiments, the electronic medical record interface application 103 may be stored and executed on a combination of the client devices 115 and the electronic medical record server 101, or by any one of the client devices 115 or electronic medical record server 101.

In some embodiments, the electronic medical record interface application 103a may be a thin-client application with some functionality executed on the client device 115a and additional functionality executed on the primary electronic medical record server 101 by the electronic medical record interface application 103b.

Health record server 107 may be either a hardware server, a software server, or a combination of software and hardware. The health record server 107 may be, or may be implemented by, a computing device including a processor, a memory, applications, a database, and network communication capabilities. The health record server 107 includes health record storage 108 configured to store health record information of a patient separately from any personally identifiable information. The health record information stored on the health record server may be linked to the personally identifiable information by a one-way hash (e.g., SHA 512) of a patient identification number. There are numerous advantages to storing patient health record information separately from personally identifiable information. For example, when stored separately from personally identifiable information, health record information may be stored unencrypted and therefore can more easily be indexed and searched. Additionally, the health record information is more secure when stored separately from personally identifiable information because a successful attack on such a system would require more than one successful breach to piece together health record information with personally identifiable information.

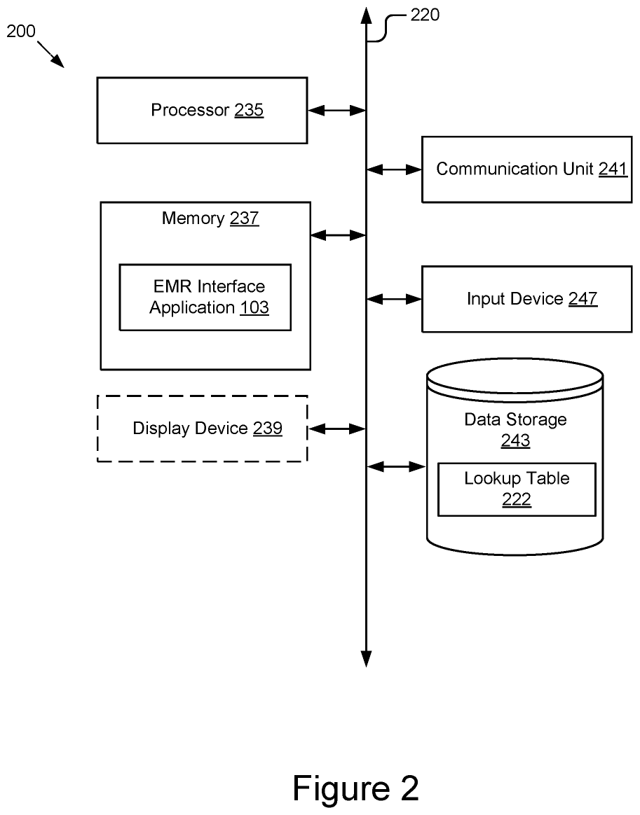

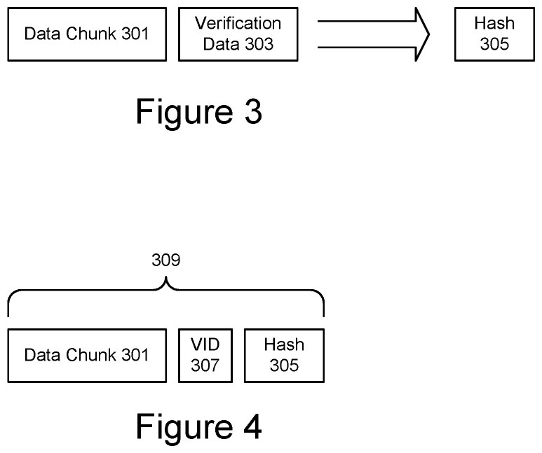

Identity provider server 109 may be either a hardware server, a software server, or a combination of software and hardware. The identity provider server 109 may be, or may be implemented by, a computing device including a processor, a memory, applications, a database, and network communication capabilities. The identity provider server 109 includes identity storage 110 configured to store personally identifiable information separately from any health record information of a patient. As described in more detail with reference to FIGS. 3-5, personally identifiable information is scrambled and stored across more than one identity provider server 109. This storage arrangement creates a more secure environment for personally identifiable information because it would take a coordinated attack across multiple physical servers to be able to retrieve and piece together any personally identifiable information of a patient. The operation of the electronic medical record interface application 103 and the functions listed above are described below in more detail below with reference to FIGS. 2-5.

FIG. 2 is a block diagram illustrating one embodiment of a computing device 200 including an electronic medical record interface application 103. The computing device 200 may also include a processor 235, a memory 237, an optional display device 239, a communication unit 241, an input device 247, and a data storage 243 according to some examples. The components of the computing device 200 are communicatively coupled by a bus 220. The bus 220 may represent one or more buses including an industry standard architecture (ISA) bus, a peripheral component interconnect (PCI) bus, a universal serial bus (USB), or some other bus known in the art to provide similar functionality. In some embodiments, the computing device 200 may be the client device 115, the electronic medical record server 101, or a combination of the client device 115 and the electronic medical record server 101.

The processor 235 may execute software instructions by performing various input/output, logical, and/or mathematical operations. The processor 235 may have various computing architectures to process data signals including, for example, a complex instruction set computer (CISC) architecture, a reduced instruction set computer (RISC) architecture, and/or an architecture implementing a combination of instruction sets. The processor 235 may be physical and/or virtual and may include a single processing unit or a plurality of processing units and/or cores. In some implementations, the processor 235 may be capable of generating and providing electronic display signals to a display device, supporting the display of images, capturing and transmitting images, performing complex tasks including various types of feature extraction and sampling, etc. In some implementations, the processor 235 may be coupled to the memory 237 via the bus 220 to access data and instructions therefrom and store data therein. The bus 220 may couple the processor 235 to the other components of the computing device 200 including, for example, the memory 237, the communication unit 241, the electronic medical record interface application 103, and the data storage 243. It will be apparent to one skilled in the art that other processors, operating systems, sensors, displays, and physical configurations are possible.

The memory 237 may store and provide access to data for the other components of the computing device 200. The memory 237 may be included in a single computing device or distributed among a plurality of computing devices as discussed elsewhere herein. In some implementations, the memory 237 may store instructions and/or data that may be executed by the processor 235. The instructions and/or data may include code for performing the techniques described herein. For example, in one embodiment, the memory 237 may store the electronic medical record interface application 103 executable by the processor 235. The memory 237 is also capable of storing other instructions and data, including, for example, an operating system, hardware drivers, other software applications, databases, etc. The memory 237 may be coupled to the bus 220 for communication with the processor 235 and the other components of the computing device 200.

The memory 237 may include one or more non-transitory computer-usable (e.g., readable, writeable) device, a static random access memory (SRAM) device, a dynamic random access memory (DRAM) device, an embedded memory device, a discrete memory device (e.g., a PROM, FPROM, ROM), a hard disk drive, an optical disk drive (CD, DVD, Blu-ray™, etc.) mediums, which can be any tangible apparatus or device that can contain, store, communicate, or transport instructions, data, computer programs, software, code, routines, etc., for processing by or in connection with the processor 235. In some implementations, the memory 237 may include one or more of volatile memory and non-volatile memory. It should be understood that the memory 237 may be a single device or may include multiple types of devices and configurations.

The display device 239 is a liquid crystal display (LCD), light emitting diode (LED) or any other similarly equipped display device, screen or monitor. The display device 239 represents any device equipped to display user interfaces, electronic images, and data as described herein. In different embodiments, the display is binary (only two different values for pixels), monochrome (multiple shades of one color), or allows multiple colors and shades. The display device 239 is coupled to the bus 220 for communication with the processor 235 and the other components of the computing device 200. It should be noted that the display device 239 is shown in FIG. 2 with dashed lines to indicate it is optional. For example, where the computing device 200 is the electronic medical record server 101, the display device 239 may not be part of the system, where the computing device 200 is the client device 115, the display device 239 is included and is used to display user interfaces for performing the techniques described herein.

The input device 247 may include any device for inputting information into the computing device 200. In some implementations, the input device 247 may include one or more peripheral devices. For example, the input device 247 may include a keyboard (e.g., a QWERTY keyboard), a pointing device (e.g., a mouse or touchpad), a microphone, an image/video capture device (e.g., camera), etc. In some implementations, the input device 247 may include a touch-screen display capable of receiving input from the one or more fingers of the user. For instance, the functionality of the input device 247 and the display 239 may be integrated, and a user of the computing device 200 (e.g., client device 115) may interact with the computing device 200 by contacting a surface of the display device 239 using one or more fingers. In this example, the user can interact with an emulated (i.e., virtual or soft) keyboard displayed on the touch-screen display device 239 by using fingers to contact the display in the keyboard regions.

The communication unit 241 is hardware for receiving and transmitting data by linking the processor 235 to the network 105 and other processing systems. The communication unit 241 receives data such as requests from the client device 115 and transmits the requests to the components of the electronic medical record interface application 103. The communication unit 241 is coupled to the bus 220. In one embodiment, the communication unit 241 may include a port for direct physical connection to the client device 115 or to another communication channel. For example, the communication unit 241 may include an RJ45 port or similar port for wired communication with the client device 115. In another embodiment, the communication unit 241 may include a wireless transceiver (not shown) for exchanging data with the client device 115 or any other communication channel using one or more wireless communication methods, such as IEEE 802.11, IEEE 802.16, Bluetooth® or another suitable wireless communication method.

In yet another embodiment, the communication unit 241 may include a cellular communications transceiver for sending and receiving data over a cellular communications network such as via short messaging service (SMS), multimedia messaging service (MMS), hypertext transfer protocol (HTTP), direct data connection, WAP, e-mail or another suitable type of electronic communication. In still another embodiment, the communication unit 241 may include a wired port and a wireless transceiver. The communication unit 241 also provides other conventional connections to the network 105 for distribution of files and/or media objects using standard network protocols such as TCP/IP, HTTP, HTTPS, and SMTP as will be understood to those skilled in the art.

The data storage 243 is a non-transitory memory that stores data for providing the functionality described herein. The data storage 243 may be coupled to the components of the EMR interface application 103 via the bus 220 to receive and provide access to data. The data storage 243 may store, among other data, lookup table 222. The lookup table 222 may be used by the electronic medical record interface application 103 to create data packages from personally identifiable information to be stored on identity provider servers 109. In the illustrated embodiment, the data storage 243 is communicatively coupled to the bus 220. The data stored in the data storage 243 is described below in more detail.

The data storage 243 may be included in the computing device 200 or in another computing device and/or storage system distinct from but coupled to or accessible by the computing device 200. The data storage 243 can include one or more non-transitory computer-readable mediums for storing the data. In some embodiments, the data storage 243 may be incorporated with the memory 237 or may be distinct therefrom. The data storage 243 may be a dynamic random access memory (DRAM) device, a static random access memory (SRAM) device, flash memory, or some other memory devices. In some embodiments, the data storage 243 also may include a non-volatile memory or similar permanent storage device and media including a hard disk drive, a floppy disk drive, a CD-ROM device, a DVD-ROM device, a DVD-RAM device, a DVD-RW device, a flash memory device, or some other mass storage device for storing information on a more permanent basis.

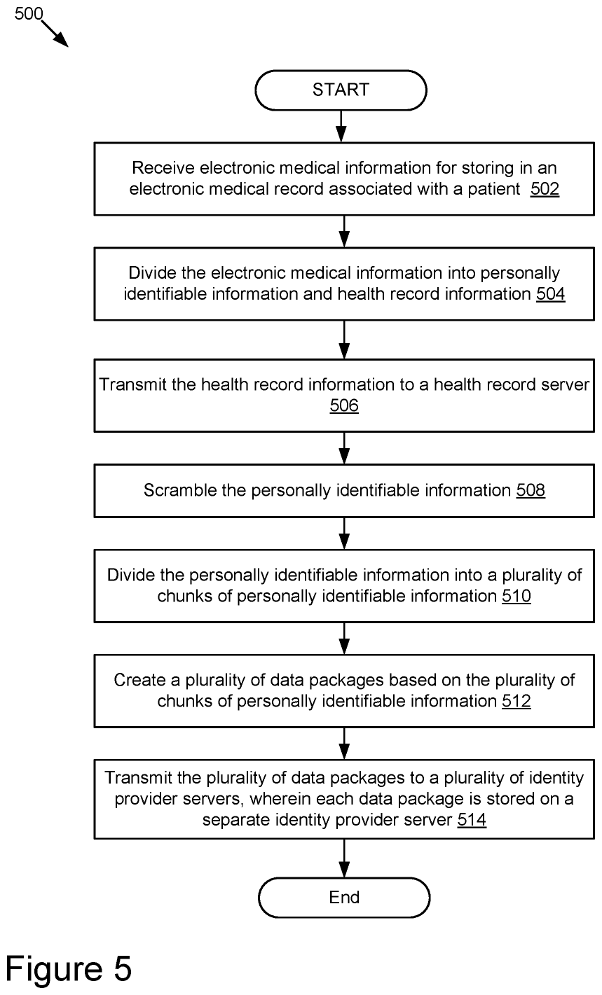

FIG. 3 is a block diagram illustrating a flow for creating a data package for storing personally identifiable information. FIG. 4 is a block diagram illustrating one embodiment of a data package for storing personally identifiable information. FIGS. 3 and 4 will be referenced in the description of FIG. 5.

FIG. 5 is a flow diagram illustrating one embodiment of an example method for securely storing electronic medical information. At 502, the electronic medical record server 101 receives electronic medical information for storing in an electronic medical record associated with a patient. The electronic medical record server 101 may receive the electronic medical information, for example, from client device 115 and/or medical device 113.

At 504, the electronic medical record interface application 103 divides the electronic medical information into personally identifiable information and health record information. For example, if the electronic medical information includes a patient name and EKG data from a medical device 113, the electronic medical record interface application 103 removes the patient's name from the EKG data.

At 506, the electronic medical record interface application 103 transmits the health record information to health record server 107, where the health record information is stored in a health record storage. In some embodiments, the health record information is stored unencrypted so that indexing, searching, and retrieval of health record information is less computationally expensive. The health record information is linked to a patient by the electronic medical record interface application 103 by associating the health record information with a one-way hash of a patient identification number. For example, the electronic medical record interface application 103 may pad the patient identification number to 64 bytes and compute a SHA512 hash of the padded patient identification number to use as a link to the personally identifiable information of the patient.

At 508, the electronic medical record interface application 103 scrambles or encrypts the personally identifiable information using an algorithmic function. Known data masking or obfuscation techniques may be used to scramble the personally identifiable information.

At 510, the electronic medical record interface application 103 divides the scrambled personally identifiable information into a plurality of chunks of personally identifiable information. At 512, the electronic medical record interface application 103 creates a plurality of data packages based on the plurality of chunks of scrambled personally identifiable information. For example, as shown in FIG. 3, the electronic medical record interface application 103 appends verification data 303 to a data chunk 301 (i.e., one of the plurality of chunks of scrambled personally identifiable information) and at 305 computes a hash 305 (e.g., using SHA256) of the data chunk 301 and the appended verification data 303. The verification data 303 is retrieved from lookup table 222 by the electronic medical record interface application 103. The lookup table may be created, for example, by generating a plurality of random numbers and associating each of the random numbers with a verification data identifier. In various embodiments, the electronic medical record interface application 103 retrieves one or more random numbers from the lookup table and appends the numbers to the data chunk 301 as verification data 303.

Once the hash 305 has been created, the electronic medical record interface application 103 appends the verification data identifiers 307 associated with the verification data that was used to create the hash 305 and the hash 305 to the data chunk. The data chunk 301, the verification data identifiers 307, and the hash 305 make up a data package 309 that is stored by the electronic medical record interface application 103 on identity provider server 109.

In some embodiments, each the plurality of data packages is stored on a separate identity provider server 109 to create redundancy and increased security. In one embodiment, the electronic medical record interface application 103, when dividing the personally identifiable information, divides the information in such a way that only a subset of the data chunks is needed to recreate the personally identifiable information (e.g., by including redundant data in the data chunks). In another embodiment, the electronic medical record interface application 103 encodes the divided personally identifiable information in such a way that only a subset of the data chunks is needed to recreate the personally identifiable information (e.g., using an erasure code or the like).

At 514, the electronic medical record interface application 103 transmits the plurality of data packages 309 to a plurality of identity provider servers. As mentioned, to improve fault tolerance and improve security each of the plurality of data packages 309 may be stored on a separate identity provider server.

While the techniques described herein relate to dividing and storing electronic medical record information, it should be understood that the reverse process may be used to retrieve personally identifiable information and related health information.

A system and method for securely storing electronic medical information has been described. In the above description, for purposes of explanation, numerous specific details are set forth in order to provide a thorough understanding of the techniques introduced above. It will be apparent, however, to one skilled in the art that the techniques can be practiced without these specific details. In other instances, structures and devices are shown in block diagram form in order to avoid obscuring the description and for ease of understanding. For example, the techniques are described in one embodiment above primarily with reference to software and particular hardware. However, the present invention applies to any type of computing system that can receive data and commands, and present information as part of any peripheral devices providing services.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

Some portions of the detailed descriptions described above are presented in terms of algorithms and symbolic representations of operations on data bits within a computer memory. These algorithmic descriptions and representations are, in some circumstances, used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing”, “computing”, “calculating”, “determining”, “displaying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

Some embodiments can take the form of an entirely hardware embodiment, an entirely software embodiment or an embodiment containing both hardware and software elements. One embodiment is implemented in software, which includes but is not limited to firmware, resident software, microcode, etc.

Furthermore, some embodiments can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A data processing system suitable for storing and/or executing program code can include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description above. In addition, the techniques are not described with reference to any particular programming language. It will be appreciated that a variety of programming languages may be used to implement the teachings of the various embodiments as described herein.

The foregoing description of the embodiments has been presented for the purposes of illustration and description. It is not intended to be exhaustive or to limit the specification to the precise form disclosed. Many modifications and variations are possible in light of the above teaching. It is intended that the scope of the embodiments be limited not by this detailed description, but rather by the claims of this application. As will be understood by those familiar with the art, the examples may be embodied in other specific forms without departing from the spirit or essential characteristics thereof. Likewise, the particular naming and division of the modules, routines, features, attributes, methodologies and other aspects are not mandatory or significant, and the mechanisms that implement the description or its features may have different names, divisions and/or formats. Furthermore, as will be apparent to one of ordinary skill in the relevant art, the modules, routines, features, attributes, methodologies and other aspects of the specification can be implemented as software, hardware, firmware or any combination of the three. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming. Additionally, the specification is in no way limited to embodiment in any specific programming language, or for any specific operating system or environment. Accordingly, the disclosure is intended to be illustrative, but not limiting, of the scope of the specification, which is set forth in the following claims.

What is claimed is:

1. A method comprising:
receiving electronic medical information for storing in an electronic medical record associated with a patient;
dividing the electronic medical information into personally identifiable information and health record information;
transmitting the health record information to a health record server;
scrambling the personally identifiable information;
dividing the personally identifiable information into a plurality of chunks of personally identifiable information;
creating a plurality of data packages based on the plurality of chunks of personally identifiable information; and
transmitting the plurality of data packages to a plurality of identity provider servers, wherein each data package is stored on a separate identity provider server.

2. The method of claim 1, further comprising:
linking the health record information to the personally identifiable information by a one-way hash of a patient identification number.

3. The method of claim 1, wherein creating the plurality of data packages comprises:
for each chunk of personally identifiable information:
appending verification data to the chunk of personally identifiable information;
creating a hash of the chunk of personally identifiable information and the verification data; and
appending the hash to the chunk of personally identifiable information.

4. The method of claim 3, further comprising retrieving the verification data from a lookup table.

5. The method of claim 4, wherein the verification data comprises a randomly generated number associated with a verification data identifier.

6. The method of claim 5, further comprising appending the verification data identifier to the chunk of personally identifiable information and the hash.

7. The method of claim 1, further comprising:
encrypting the plurality of data packages such that only a subset of the plurality of data packages are required to recreate the personally identifiable information.

8. A system comprising:
one or more processors; and
a memory, the memory storing instructions, which when executed cause the one or more processors to:
receive electronic medical information for storing in an electronic medical record associated with a patient;
divide the electronic medical information into personally identifiable information and health record information;
transmit the health record information to a health record server;
scramble the personally identifiable information;
divide the personally identifiable information into a plurality of chunks of personally identifiable information;
create a plurality of data packages based on the plurality of chunks of personally identifiable information; and
transmit the plurality of data packages to a plurality of identity provider servers,
wherein each data package is stored on a separate identity provider server.

9. The system of claim 8, wherein the instructions further cause the one or more processors to:
link the health record information to the personally identifiable information by a one-way hash of a patient identification number.

10. The system of claim 8, wherein to create the plurality of data packages, the instructions further cause the one or more processors to:
for each chunk of personally identifiable information:
append verification data to the chunk of personally identifiable information;
create a hash of the chunk of personally identifiable information and the verification data; and
append the hash to the chunk of personally identifiable information.

11. The system of claim 10, wherein the instructions further cause the one or more processors to retrieve the verification data from a lookup table.

12. The system of claim 11, wherein the verification data comprises a randomly generated number associated with a verification data identifier.

13. The system of claim 12, wherein the instructions further cause the one or more processors to append the verification data identifier to the chunk of personally identifiable information and the hash.

14. The system of claim 8, wherein the instructions further cause the one or more processors to:
encrypt the plurality of data packages such that only a subset of the plurality of data packages are required to recreate the personally identifiable information.

15. A computer program product comprising a non-transitory computer readable medium storing a computer readable program, wherein the computer readable program when executed on a computer causes the computer to:
receive electronic medical information for storing in an electronic medical record associated with a patient;
divide the electronic medical information into personally identifiable information and health record information;
transmit the health record information to a health record server;
scramble the personally identifiable information;
divide the personally identifiable information into a plurality of chunks of personally identifiable information;
create a plurality of data packages based on the plurality of chunks of personally identifiable information; and
transmit the plurality of data packages to a plurality of identity provider servers,
wherein each data package is stored on a separate identity provider server.

16. The computer program product of claim 15, wherein the computer readable program further causes the computer to:
link the health record information to the personally identifiable information by a one-way hash of a patient identification number.

17. The computer program product of claim 15, wherein to create the plurality of data packages, the computer readable program further causes the computer to:
for each chunk of personally identifiable information:
append verification data to the chunk of personally identifiable information;
create a hash of the chunk of personally identifiable information and the verification data; and
append the hash to the chunk of personally identifiable information.

18. The computer program product of claim 17, wherein the verification data comprises a randomly generated number associated with a verification data identifier.

19. The computer program product of claim 18, wherein the computer readable program further causes the computer to append the verification data identifier to the chunk of personally identifiable information and the hash.

20. The computer program product of claim 19, wherein the computer readable program further causes the computer to:
encrypt the plurality of data packages such that only a subset of the plurality of data packages are required to recreate the personally identifiable information.
